(19) United States
(12) Patent Application Publication
(10) Pub. No.: US 2017/0056496 A1
(43) Pub. Date: Mar. 2, 2017

(54) ANTITUMOR COMBINATIONS CONTAINING ANTIBODIES RECOGNIZING SPECIFICALLY CD38 AND CYCLOPHOSPHAMIDE

(71) Applicant: Sanofi, Paris (FR)

(72) Inventors: Pascale LEJEUNE, Paris (FR); Patricia VRIGNAUD, Paris (FR)

(21) Appl. No.: 15/235,427

(22) Filed: Aug. 12, 2016

Related U.S. Application Data
(62) Division of application No. 13/131,389, filed on Aug. 31, 2011, now abandoned.

(30) Foreign Application Priority Data
Nov. 28, 2008 (EP) 08291119.9

(51) Int. Cl. A61K 39/395; A61K 31/664

(52) U.S. Cl. CPC A61K 39/3955; A61K 31/664; A61K 2039/505

(57) ABSTRACT

Pharmaceutical composition comprising an antibody specifically recognizing CD38 and cyclophosphamide.

### CROSS-REFERENCE TO RELATED APPLICATIONS

This application is a division of U.S. patent application Ser. No. 13/131,389, filed Aug. 31, 2011, which is a National Stage filing under 35 U.S.C. §371 of International Patent Application No. PCT/IB2009/055392, filed Nov. 27, 2009, which claims the benefit of priority of European Patent Application No. 08291119.9, filed Nov. 28, 2008, the entire contents of which are hereby incorporated herein by reference in their entireties.

The present invention relates to combinations of monoclonal antibodies directed against CD38 and cyclophosphamide known under the trade-name cytoxan (or neosar) which are therapeutically useful in the treatment of neoplastic diseases.

CD38 is a 45 kD type II transmembrane glycoprotein with a long C-terminal extracellular domain and a short N-terminal cytoplasmic domain. The CD38 protein is a bifunctional ectoenzyme that can catalyze the conversion of NAD⁺ into cyclic ADP-ribose (cADPR) and also hydrolyze cADPR into ADP-ribose. CD38 is upregulated and has been implicated in many hematopoietic malignancies.

Monoclonal antibodies 38SB13, 38SB18, 38SB19, 38SB30, 38SB31, and 38SB39, which specifically recognize CD38, are described in PCT application WO2008/047242. Said anti-CD38 antibodies are capable of killing CD38⁺ cells by three different cytotoxic mechanisms, induction of apoptosis, antibody-dependent cell-mediated cytotoxicity (ADCC), and complement-dependent cytotoxicity (CDC). In addition, these antibodies are able to directly induce apoptosis of CD38⁺ cells, even without the presence of stroma cells or stroma-derived cytokines. Cyclophosphamide is an alkylating agent used in chemotherapy. There is still a need for novel and efficacious medicaments which can be used in cancer therapy.

It has now been found, and for this invention, that the efficacy of the humanized anti-CD38 antibodies may be considerably improved when it is administered in combination with at least one substance which is therapeutically useful in anticancer treatments and has a mechanism identical to or different from the one of the humanized anti-CD38 antibodies and which is limited in the present invention to cyclophosphamide.

The term “antibody” is used herein in the broadest sense and specifically covers monoclonal antibodies (including full length monoclonal antibodies) of any isotype such as IgG, IgM, IgA, IgD and IgE, polyclonal antibodies, multispecific antibodies, chimeric antibodies, and antibody fragments. A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions outside of the CDRs are called the “framework regions”.

As used herein, “V_{H}” or “VH” refers to the variable region of an immunoglobulin heavy chain of an antibody, including the heavy chain of an Fv, scFv, dsFv, Fab, Fab′ or F(ab′)2 fragment. Reference to “V_{L}” or “VL” refers to the variable region of the immunoglobulin light chain of an antibody, including the light chain of an Fv, scFv, dsFv, Fab, Fab′ or F(ab′)2 fragment.

The 38SB13 antibody comprises at least one heavy chain having an amino acid sequence consisting of SEQ ID NO: 50 and at least one light chain having an amino acid sequence consisting of SEQ ID NO: 38, said heavy chain comprising three sequential CDRs having amino acid sequences consisting of SEQ ID NOS: 1, 2, and 3, and said light chain comprising three sequential CDRs having amino acid sequences consisting of SEQ ID NOS: 4, 5, and 6.

The 38SB18 antibody comprises at least one heavy chain having an amino acid sequence consisting of SEQ ID NO: 52 and at least one light chain having an amino acid sequence consisting of SEQ ID NO: 40, said heavy chain comprising three sequential CDRs having amino acid sequences consisting of SEQ ID NOS: 7, 8, and 9, and said light chain comprising three sequential CDRs having amino acid sequences consisting of SEQ ID NOS: 10, 11, and 12.

The 38SB19 antibody comprises at least one heavy chain having an amino acid sequence consisting of SEQ ID NO: 54 and at least one light chain having an amino acid sequence consisting of SEQ ID NO: 42, said heavy chain comprising three sequential CDRs having amino acid sequences consisting of SEQ ID NOS: 13, 14, and 15, and said light chain comprising three sequential CDRs having amino acid sequences consisting of SEQ ID NOS: 16, 17, and 18.

The 38SB30 antibody comprises at least one heavy chain having an amino acid sequence consisting of SEQ ID NO: 56 and at least one light chain having an amino acid sequence consisting of SEQ ID NO: 44, said heavy chain comprising three sequential CDRs having amino acid sequences consisting of SEQ ID NOS: 19, 20, and 21, and said light chain comprising three sequential CDRs having amino acid sequences consisting of SEQ ID NOS: 22, 23, and 24.

The 38SB31 antibody comprises at least one heavy chain having an amino acid sequence consisting of SEQ ID NO: 58 and at least one light chain having an amino acid sequence consisting of SEQ ID NO: 46, said heavy chain comprising three sequential CDRs having amino acid sequences consisting of SEQ ID NOS: 25, 26, and 27, and said light chain comprising three sequential CDRs having amino acid sequences consisting of SEQ ID NOS: 28, 29, and 30.

The 38SB39 antibody comprises at least one heavy chain having an amino acid sequence consisting of SEQ ID NO: 60 and at least one light chain having an amino acid sequence consisting of SEQ ID NO: 48, said heavy chain comprising three sequential CDRs having amino acid sequences consisting of SEQ ID NOS: 31, 32, and 33, and said light chain comprising three sequential CDRs having amino acid sequences consisting of SEQ ID NOS: 34, 35, and 36.

The hybridoma cell lines producing the 38SB13, 38SB18, 38SB19, 38SB30, 38SB31, and 38SB39 murine anti-CD38 antibodies have been deposited at the American Type Culture Collection (10801 University Bld, Manassas, Va., 20110-2209, USA), on Jun. 21, 2006, under the deposit numbers PTA-7667, PTA-7669, PTA-7670, PTA-7666, PTA-7668, and PTA-7671, respectively (as described in WO2008/047242).

The term “humanized antibody”, as used herein, refers to a chimeric antibody which contain minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modelling, statistical analysis and mutagenesis to alter the non-CDR surfaces of antibody variable regions to resemble the surfaces of known antibodies of the target host. The CDR grafting technology involves substituting the complementarity determining regions of, for example, a mouse antibody, into a human framework domain, e.g., see WO 92/22653. Humanized chimeric antibodies preferably have constant regions and variable regions other than the complementarity determining regions (CDRs) derived substantially or exclusively from the corresponding human antibody regions and CDRs derived substantially or exclusively from a mammal other than a human.

Strategies and methods for the resurfacing of antibodies, and other methods for reducing immunogenicity of antibodies within a different host, are disclosed in US Patent 5,639,641, which is hereby incorporated in its entirety by reference. Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5): 489-498; Studnicka G. M. et al., 1994, Protein Engineering, 7(6): 805-814; Roguska M. A. et al., 1994, PNAS, 91: 969-973), chain shuffling (U.S. Pat. No. 5,565,332), and identification of flexible residues (PCT/US2008/074381). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

The anti-CD38 antibodies of the pharmaceutical combination of the present invention are humanized antibodies which recognize CD38 and kill CD38⁺ cells by apoptosis, ADCC, and CDC. In a further embodiment, the humanized antibodies of the invention are capable of killing said CD38⁺ cells by apoptosis even in the absence of stroma cells or stroma-derived cytokines.

A preferred embodiment of such a humanized antibody is a humanized 38SB13, 38SB18, 38SB19, 38SB30, 38SB31, or 38SB39 antibody, or an epitope-binding fragment thereof.

The CDRs of the 38SB13, 38SB18, 38SB19, 38SB30, 38SB31, and 38SB39 antibodies are identified by modelling and their molecular structures have been predicted. Thus, in one embodiment, this invention provides humanized antibodies or epitope-binding fragment thereof comprising one or more CDRs having an amino acid sequence selected from the group consisting of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 81. In a preferred embodiment, a humanized version of 38SB13 is provided, which comprises at least one heavy chain and at least one light chain, wherein said heavy chain comprises three sequential complementarity-determining regions having amino acid sequences represented by SEQ ID NOS: 1, 2, and 3, and wherein said light chain comprises three sequential complementarity-determining regions having amino acid sequences represented by SEQ ID NOS: 4, 5, and 6. In another preferred embodiment, a humanized version of 38SB18 is provided, which comprises at least one heavy chain and at least one light chain, wherein said heavy chain comprises three sequential complementarity-determining regions having amino acid sequences represented by SEQ ID NOS: 7, 8, and 9, and wherein said light chain comprises three sequential complementarity-determining regions having amino acid sequences represented by SEQ ID NOS: 10, 11, and 12. In another preferred embodiment, a humanized version of 38SB19 is provided, which comprises at least one heavy chain and at least one light chain, wherein said heavy chain comprises three sequential complementarity-determining regions having amino acid sequences represented by SEQ ID NOS: 13, 81, and 15, and wherein said light chain comprises three sequential complementarity-determining regions having amino acid sequences represented by SEQ ID NOS: 16, 17, and 18. In another preferred embodiment, a humanized version of 38SB30 is provided, which comprises at least one heavy chain and at least one light chain, wherein said heavy chain comprises three sequential complementarity-determining regions having amino acid sequences represented by SEQ ID NOS: 19, 20, and 21, and wherein said light chain comprises three sequential complementarity-determining regions having amino acid sequences represented by SEQ ID NOS: 22, 23, and 24. In another preferred embodiment, a humanized version of 38SB31 is provided, which comprises at least one heavy chain and at least one light chain, wherein said heavy chain comprises three sequential complementarity-determining regions having amino acid sequences represented by SEQ ID NOS: 25, 26, and 27, and wherein said light chain comprises three sequential complementarity-determining regions having amino acid sequences represented by SEQ ID NOS: 28, 29, and 30. In another preferred embodiment, a humanized version of 38SB39 is provided, which comprises at least one heavy chain and at least one light chain, wherein said heavy chain comprises three sequential complementarity-determining regions having amino acid sequences represented by SEQ ID NOS: 31, 32, and 33, and wherein said light chain comprises three sequential complementarity-determining regions having amino acid sequences represented by SEQ ID NOS: 34, 35, and 36.

In one embodiment, this invention provides humanized antibodies or fragments thereof which comprise a V_{H} having an amino acid sequence selected from the group of SEQ ID NOS: 66 and 72. In a preferred embodiment, a humanized 38SB19 antibody is provided which comprises a V_{H} having an amino acid sequence represented by SEQ ID NO: 66. In another preferred embodiment, a humanized 38SB31 antibody is provided which comprises a V_{H} having an amino acid sequence represented by SEQ ID NO: 72.

In another embodiment, this invention provides humanized antibodies or fragments thereof which comprise a V_{L} having an amino acid sequence selected from the group of SEQ ID NOS: 62, 64, 68, and 70. In a preferred embodiment, a humanized 38SB19 antibody is provided which comprises a V_{L} having an amino acid sequence chosen from the group of SEQ ID NOS: 62 and 64. In another preferred embodiment, a humanized 38SB31 antibody is provided which comprises a V_{L} having an amino acid sequence chosen from the group of SEQ ID NOS: 68 and 70.

Each of the humanized versions of the 38SB13, 38SB18, 38SB19, 38SB30, 38SB31, and 38SB39 antibodies has been shown to be particularly advantageous as an anticancer agent. The preparation, physical properties and beneficial pharmacological properties thereof are described in WO 2008/047242, which is incorporated by reference herein in its entirety. Generally, the doses used for treating human beings, which depend on factors distinctive to the subject to be treated, are between 1 and 150 mg/kg administered orally or between 1 and 150 mg/kg administered intravenously.

Cyclophosphamide (cytoxan®) is a nitrogen mustard alkylating agent used to treat various cancers. It is a pro-drug that is converted in the liver to active forms that have chemotherapeutic activity. As an alkylating agent, cyclophosphamide prevents cell division primarily by crosslinking DNA strands, ultimately resulting in cell death. Cyclophosphamide is available in both oral and parental formulations.

One aspect of the invention is a pharmaceutical composition comprising an anti-CD38 antibody in combination with at least cyclophosphamide. Since the activity of the products depends on the doses used, it is thus possible to use lower doses and to increase the activity while decreasing the toxicity phenomena. The improved efficacy of a combination according to the invention may be demonstrated by determination of the therapeutic synergy. A combination manifests therapeutic synergy if it is therapeutically superior to the best agent of the study used alone at its maximum tolerated dose or at its highest dose tested when toxicity cannot be reached in the animal species.

This efficacy may be quantified, for example, by the log₁₀ cell kill, which is determined according to the following formula:

log₁₀ cell kill=T−C(days)/3.32×T_{d}

in which T−C represents the tumor growth delay, which is the median time in days for the tumors of the treated group (T) and the tumors of the control group (C) to have reached a predetermined value (1 g for example), and T_{d} represents the time in days needed for the volume of the tumor to double in the control animals [T. H. Corbett et al., Cancer, 40: 2660-2680 (1977); F. M. Schabel et al., Cancer Drug Development, Part B, Methods in Cancer Research, 17: 3-51, New York, Academic Press Inc. (1979)]. A product is considered to be active if log₁₀ cell kill is greater than or equal to 0.7. A product is considered to be very active if log₁₀ cell kill is greater than or equal to 2.8.

In some cases, when treatment duration is over 10 days and differs between the 2 compounds evaluated in the combination, a log cell kill net is calculated:

log₁₀ cell kill net=(T−C)−treatment duration/3.32×T_{d}.

Activity is declared when log cell kill net is greater than or equal to 0.

The combination will manifest therapeutic synergy when the log₁₀ cell kill is greater than the value of the log₁₀ cell kill of the best constituent administered alone at its maximum tolerated dose or at its highest dose tested.

The efficacy of the combinations on solid tumors may be determined experimentally in the following manner:

The animals subjected to the experiment, generally mice, are subcutaneously grafted bilaterally with 30 to 60 mg of a tumor fragment on day 0. The animals bearing tumors are randomized based on their tumor size before being subjected to the various treatments and controls. Chemotherapy begins when tumors have reached a predetermined size after grafting, depending on the type of tumor, and the animals are observed every day. The different animal groups are weighed daily during treatment until the maximum weight loss is reached and subsequent full weight recovery has occurred. The groups are then weighed once or twice a week until the end of the trial.

The tumors are measured 1 to 5 times a week, depending on the tumor doubling time, until the tumor reaches approximately 2 g, or until the animal dies (if this occurs before the tumor reaches 2 g). The animals are necropsied immediately after euthanasia or death.

The antitumor activity is determined in accordance with the different parameters recorded.

Results obtained with combinations of hu38SB19 and cyclophosphamide used at their optimal doses are indicated hereunder as examples.

The present invention also relates, therefore, to pharmaceutical compositions containing the combinations according to the invention.

The constituents of which the combination are composed may be administered simultaneously, semi-simultaneously, separately, or spaced out over a period of time so as to obtain the maximum efficacy of the combination; it being possible for each administration to vary in its duration from a rapid administration to a continuous perfusion.

As a result, for the purposes of the present invention, the combinations are not exclusively limited to those which are obtained by physical association of the constituents, but also to those which permit a separate administration, which can be simultaneous or spaced out over a period of time.

The compositions according to the invention are preferably compositions which can be administered parentally. However, these compositions may be administered orally, subcutaneously or intraperitoneally in the case of localized regional therapies.

The compositions for parental administration are generally pharmaceutically acceptable, sterile solutions or suspensions which may optionally be prepared as required at the time of use. For the preparation of non-aqueous solutions or suspensions, natural vegetable oils such as olive oil, sesame oil or liquid petroleum or injectable organic esters such as ethyl oleate may be used. The sterile aqueous solutions can consist of a solution of the product in water. The aqueous solutions are suitable for intravenous administration provided the pH is appropriately adjusted and the solution is made isotonic, for example with a sufficient amount of sodium chloride or glucose. The sterilization may be carried out by heating or by any other means which does not adversely affect the composition. The combinations may also take the form of liposomes or the form of an association with carriers as cyclodextrins or polyethylene glycols.

The compositions for oral, subcutaneous or intraperitoneal administration are preferably aqueous suspensions or solutions.

In the combinations according to the invention, the application of the constituents of which may be simultaneous, separate or spaced out over a period of time, it is especially advantageous for the amount of humanized anti-CD38 antibody to represent from 10 to 90% by weight of the combination, it being possible for this content to vary in accordance with the nature of the associated substance, the efficacy sought and the nature of the cancer to be treated.

The combinations according to the invention are especially useful in the treatment of several types of cancers including (but not limited to) the following: carcinomas and adenocarcinomas, including that of the bladder, breast, colon, head-and-neck, prostate, kidney, liver, lung, ovary, pancreas, stomach, cervix, thyroid and skin, and including squamous cell carcinoma; hematopoietic tumors of lymphoid lineage, including multiple myeloma, leukemia, acute and chronic lymphocytic (or lymphoid) leukemia, acute and chronic lymphoblastic leukemia, B-cell lymphoma, T-cell lymphoma, non-Hodgkin lymphoma (e.g. Burkitt's lymphoma); hematopoietic tumors of myeloid lineage, including acute and chronic myelogenous (myeloid or myelocytic) leukemias, and promyelocytic leukemia; tumors of mesenchymal origin, including fibrosarcoma, osteosarcoma and rhabdomyosarcoma; tumors of the central and peripheral nervous system, including astrocytoma, neuroblastoma, glioma, and schwannomas; and other tumors, including melanoma, teratocarcinoma, xeroderma pigmentosum, keratoacanthoma, and seminoma, and other cancers yet to be determined in which CD38 is expressed. They are mainly useful for treating leukemia, lymphoma and cancers resistant to the commonly used anticancer agents as the anti-CD38 antibodies of the invention have a unique mechanism of action.

Thus, the invention also encompasses the use of the above combinations for the manufacture of a medicament for the treatment of cancer.

### EXAMPLE

In this example, the effectiveness of an anti-CD38 antibody/cyclophosphamide combination of the invention for tumor growth inhibition was demonstrated in vivo.

The selected tumor model was a transplantable human Burkitt's lymphoma cell line, the Namalwa model, implanted in SCID mice.

Hu38SB19 was formulated in glucose 5% in water. Hu38SB19, as single agent, was administered intravenously, twice a week, between days 11 and 18 post-tumor implantation. In the combination arm, hu38SB19 treatment was pursued until day 35 (except for the highest dose of the combination for which treatment was stopped when toxicity was reached on day 18).

Cyclophosphamide was formulated in glucose 5% in water. Cyclophosphamide was administered intravenously, once per day, on day 11 and 15 after tumor implantation.

The results of the experiment are reported in Table 1.

Tumor doubling time=1.9 days.

The following end points have been used:
Toxicity was declared at dosages inducing ≧20% body weight loss or 10% drug death,
Antitumor efficacy was determinated by calculating log10 cell kill gross=(T−C)/[3.32×(tumor doubling time in days)]
(T meaning the median time of the treated mice to reach 1000 mg and C the median time (15.7 days) of the control mice to reach the same size; tumor-free survivors are excluded from these calculations and are tabulated separately). No antitumor activity was declared for log cell kill gross <0.7, and the treatment was declared highly active for log cell kill ≧2.8
Tumor Free Survivors (TFS): correspond to complete regression below the limit of palpation (63 mg) for the entire duration of the study (>100 days post last treatment).
Therapeutic Synergism: a combination has therapeutic synergism if it is more active than the best single agent of the study (by at least 1 log cell kill).

Toxicity for cyclophosphamide alone was observed at a dose of 286.1 mg/kg/injection, with 3 deaths out of 5 mice (60%). Thus the highest nontoxic dose (HNTD) for cyclophosphamide was 177.4 mg/kg/inj (total injected dose=354.8 mg/kg). The 177.4 mg/kg/inj dose was found to be highly active with a log cell kill gross of 4.8.

Regarding hu38SB19, the product was well tolerated at a dose of 40 mg/kg/inj (total dose of 120.0 mg/kg). No toxicity was observed, which can be explained by the lack of cross-reactivity of the antibody with murine CD38. The log cell kill was 0.4, indicating that hu38DB19 was not active under these conditions.

The combination of cyclophosphamide at 286.1 mg/kg/inj and hu38SB19 at 40 mg/kg/inj was toxic, with 3 drug-related deaths out of 6 animals (50%), i.e. very similar to what was observed with cyclophosphamide alone at the same dose. The dose of 177.4 mg/kg/inj of cyclophosphamide with 40 mg/kg/inj of hu38SB19 was considered to be the HNTD. This dose displayed a log cell kill of 20.0 and 1/6 TFS (day 196) and was thus considered highly active. Even the lower doses of 110.0 mg/kg/inj and 68.2 mg/kg/inj of cyclophosphamide with 40 mg/kg/inj of hu38SB19 were highly active, with log cell kill gross of 15.7 and 6.9, respectively. Remarkably, the antitumor activity of the combination, at all three dose levels evaluated, was greater by more than one log cell kill than the one observed for the best agent, cyclophosphamide (4.8 log cell kill at the HNTD). We conclude that this combination shows a therapeutic synergism. This therapeutic synergism is also clearly established using the log cell kill net value taking into account the treatment duration (16 log cell kill net for the combination versus 4 log cell kill net for cyclophosphamide alone).

TABLE I
Combination of hu38SB19 and cyclophosphamide (CPA) against advanced human Burkitt's lymphoma Namalwa implanted
in SCID female mice.

| | | | T-C | | |
|---|---|---|---|---|---|
| agent, schedule and dose in mg/kg/inj | | % BWC at nadir | in days | log₁₀ cell kill gross/ | |
| (total dose) | | (day) | (1000 mg) | net | Comments |
| hu38SB19, IV | CPA, IV | +5.4 (19) | 2.5 | 0.4/−0.9 | HDT - inactive |
| Day 11-35, twice a week | Day 11, 15 | | | | |
| 40.0 (120.0)* | — | | | | |
| — | 286.1 (572.2) | −21.2 (20) | — | — | Toxic ⅗ deaths |
| — | 177.4 (354.8) | −11.1 (19) | 30.1 | 4.8/4.0 | HNTD, highly active |
| — | 110.0 (220.0) | −5.2 (17) | 19.8 | 3.1/2.3 | Highly active |
| — | 68.2 (136.4) | −3.8 (18) | 9.8 | 1.6/0.8 | Active |
| 40.0 (120.0)* | 286.1 (572.2) | −21.7 (21) | — | — | Toxic 3/6 deaths |
| 40.0 (320.0) | 177.4 (354.8) | −8.0 (16) | 126.1 | 20.0/16.0 | HNTD, highly active ⅙ TFS |
| 40.0 (320.0) | 110.0 (220.0) | −4.2 (17) | 99.3 | 15.7/11.8 | Highly active |
| 40.0 (320.0) | 68.2 (136.4) | −4.6 (29) | 43.7 | 6.9/3.0 | Highly active |

Tumor doubling time = 1.9 days. Median tumor size at start of therapy = 142-149 mg. Time for median tumor to reach 1000 mg = 15.7 days.
Formulations: hu38SB19 and CPA = glucose 5% in water.
BWC = body weight change,
T-C = tumor growth delay,
HNTD = highest nontoxic dose,
HDT = highest dose tested,
CPA = cyclophosphamide,
TFS = tumor free survivors,
IV = intravenous.
*Treament was stopped on day 18.

1-10. (canceled)

11. A method for treating a subject with cancer comprising administering to the subject a pharmaceutical combination comprising cyclophosphamide and an antibody that specifically binds to CD38, wherein the antibody comprises at least one heavy chain comprising three sequential complementarity-determining regions (CDRs) represented by SEQ ID NOs: 13, 81, and 15, and at least one light chain comprising three sequential CDRs represented by SEQ ID NOs: 16, 17 and 18, and wherein the antibody and cyclophosphamide constituents of the combination are physically separate, thereby treating cancer in the subject.

12. The method of claim 11, wherein said antibody is capable of killing a CD38+ cell by apoptosis, antibody-dependent cell-mediated cytotoxicity (ADCC), and complement-dependent cytotoxicity (CDC).

13. The method of claim 11, wherein the antibody is a chimeric antibody or a humanized antibody.

14. The method of claim 11, wherein the heavy chain comprises the amino acid sequence of SEQ ID NO: 66, and wherein the light chain comprises the amino acid sequence of SEQ ID NOs: 62 or 64.

15. The method of claim 11, wherein the antibody comprises the heavy chain and light chain of an antibody produced by the hybridoma cell line deposited at the ATCC and having a deposit number of PTA-7670.

16. The method of claim 11, wherein the antibody and cyclophosphamide constituents of the combination are administered simultaneously.

17. The method of claim 11, wherein the antibody and cyclophosphamide constituents of the combination are administered spaced out over a period of time.

18. The method of claim 11, wherein the cancer is a hematological cancer.

19. The method of claim 18, wherein the cancer is non-Hodgkin lymphoma.

20. The method of claim 18, wherein the cancer is Burkitt's lymphoma.

21. The method of claim 18, where the cancer is multiple myeloma.

22. A method for treating a subject with cancer comprising administering to the subject a pharmaceutical combination comprising cyclophosphamide and an antibody that specifically binds to CD38, wherein the antibody comprises at least one heavy chain comprising the amino acid sequence of SEQ ID NO:66, and at least one light chain comprising the amino acid sequence of SEQ ID NO:62, and wherein the antibody and cyclophosphamide constituents of the combination are physically separate, thereby treating cancer in the subject.

23. The method of claim 22, wherein said antibody is capable of killing a CD38+ cell by apoptosis, antibody-dependent cell-mediated cytotoxicity (ADCC), and complement-dependent cytotoxicity (CDC).

24. The method of claim 22, wherein the antibody and cyclophosphamide constituents of the combination are administered simultaneously.

25. The method of claim 22, wherein the antibody and cyclophosphamide constituents of the combination are administered spaced out over a period of time.

26. The method of claim 22, wherein the cancer is a hematological cancer.

27. The method of claim 26, wherein the cancer is non-Hodgkin lymphoma.

28. The method of claim 26, wherein the cancer is Burkitt's lymphoma.

29. The method of claim 26, where the cancer is multiple myeloma.


SEQUENCE LISTING (81 sequences)

SEQ ID NO: 1 - protein, 5 aa, Mus sp.
SYGMN

SEQ ID NO: 2 - protein, 17 aa, Mus sp.
WINTYTGEPTYADDFKG

SEQ ID NO: 3 - protein, 5 aa, Mus sp.
RGFAY

SEQ ID NO: 4 - protein, 15 aa, Mus sp.
RASESVEIYGNGFMN

SEQ ID NO: 5 - protein, 7 aa, Mus sp.
RASNLES

SEQ ID NO: 6 - protein, 9 aa, Mus sp.
QQINEDPFT

SEQ ID NO: 7 - protein, 5 aa, Mus sp.
NSGMN

SEQ ID NO: 8 - protein, 17 aa, Mus sp.
WINTYTGEPTYADDFKG

SEQ ID NO: 9 - protein, 5 aa, Mus sp.
RGFVY

SEQ ID NO: 10 - protein, 15 aa, Mus sp.
RASESVAIYGNSFLK

SEQ ID NO: 11 - protein, 7 aa, Mus sp.
RASNLES

SEQ ID NO: 12 - protein, 9 aa, Mus sp.
QQINEDPYT

SEQ ID NO: 13 - protein, 5 aa, Mus sp.
DYWMQ

SEQ ID NO: 14 - protein, 17 aa, Mus sp.
TIYPGDGDTGYAQKFKG

SEQ ID NO: 15 - protein, 11 aa, Mus sp.
GDYYGSNSLDY

SEQ ID NO: 16 - protein, 11 aa, Mus sp.
KASQDVSTVVA

SEQ ID NO: 17 - protein, 7 aa, Mus sp.
SASYRYI

SEQ ID NO: 18 - protein, 9 aa, Mus sp.
QQHYSPPYT

SEQ ID NO: 19 - protein, 5 aa, Mus sp.
GSWMN

SEQ ID NO: 20 - protein, 17 aa, Mus sp.
RIYPGDGDIIYNGNFRD

SEQ ID NO: 21 - protein, 10 aa, Mus sp.
WGTFTPSFDY

SEQ ID NO: 22 - protein, 11 aa, Mus sp.
KASQDVVTAVA

SEQ ID NO: 23 - protein, 7 aa, Mus sp.
SASHRYT

SEQ ID NO: 24 - protein, 9 aa, Mus sp.
QQHYTTPTT

SEQ ID NO: 25 - protein, 5 aa, Mus sp.
SYTLS

SEQ ID NO: 26 - protein, 17 aa, Mus sp.
TISIGGRYTYYPDSVEG

SEQ ID NO: 27 - protein, 8 aa, Mus sp.
DFNGYSDF

SEQ ID NO: 28 - protein, 11 aa, Mus sp.
KASQVVGSAVA

SEQ ID NO: 29 - protein, 7 aa, Mus sp.
WASTRHT

SEQ ID NO: 30 - protein, 9 aa, Mus sp.
QQYNSYPYT

SEQ ID NO: 31 - protein, 5 aa, Mus sp.
NFGMH

SEQ ID NO: 32 - protein, 17 aa, Mus sp.
YIRSGSGTIYYSDTVKG

SEQ ID NO: 33 - protein, 11 aa, Mus sp.
SYYDFGAWFAY

SEQ ID NO: 34 - protein, 11 aa, Mus sp.
KASQNVGTNVA

SEQ ID NO: 35 - protein, 7 aa, Mus sp.
SASSRYS

SEQ ID NO: 36 - protein, 9 aa, Mus sp.
QQYNSYPLT

SEQ ID NO: 37 - DNA, 336 bp, Mus sp.
  CDS (1)..(336)
aacattgtgctgacccaatctccagcttctttggctgtgtctcttgggcagagggccaccatatcctgcagagccagtgaaagtgttgagatttatggcaatggttttatgaactggttccagcagaaaccaggacagccacccaaactcctcatctatcgtgcatccaacctagaatctgggatccctgccaggttcagtggcagtgggtctaggacagagttcaccctcaccattgatcctgtggaggctgatgatgttgcaacctattactgtcaacaaattaatgaggatccattcacgttcggctcggggacaaagttggaaataaaacgg

SEQ ID NO: 38 - protein, 112 aa, Mus sp.
NIVLTQSPASLAVSLGQRATISCRASESVEIYGNGFMNWFQQKPGQPPKLLIYRASNLESGIPARFSGSGSRTEFTLTIDPVEADDVATYYCQQINEDPFTFGSGTKLEIKR

SEQ ID NO: 39 - DNA, 336 bp, Mus sp.
  CDS (1)..(336)
gacattgtactgacccaatctccagcttctttggctgtgtctctagggcagagggccaccatatcctgcagagccagtgagagtgttgctatttatggcaatagttttctgaaatggttccagcagaaaccgggacagccacccaaactcctcatctatcgtgcatccaacctagaatctgggatccctgccaggttcagtggcagtgggtctgggacagacttcaccctcaccattaatcctgtggaggctgatgatgttgcaacctattactgtcagcaaattaatgaggatccgtacacgttcggaggggggaccaagctggaaataaaacgg

SEQ ID NO: 40 - protein, 112 aa, Mus sp.
DIVLTQSPASLAVSLGQRATISCRASESVAIYGNSFLKWFQQKPGQPPKLLIYRASNLESGIPARFSGSGSGTDFTLTINPVEADDVATYYCQQINEDPYTFGGGTKLEIKR

SEQ ID NO: 41 - DNA, 324 bp, Mus sp.
  CDS (1)..(324)
gacattgtgatggcccagtctcacaaattcatgtccacatcagttggagacagggtcagcatcacctgcaaggccagtcaggatgtgagtactgttgtggcctggtatcaacagaaaccaggacaatctcctaaacgactgatttactcggcatcctatcggtatattggagtccctgatcgcttcactggcagtggatctgggacggatttcactttcaccatcagcagtgtgcaggctgaagacctggcagtttattactgtcagcaacattatagtcctccgtacacgttcggaggggggaccaagctggaaataaaacgg

SEQ ID NO: 42 - protein, 108 aa, Mus sp.
DIVMAQSHKFMSTSVGDRVSITCKASQDVSTVVAWYQQKPGQSPKRLIYSASYRYIGVPDRFTGSGSGTDFTFTISSVQAEDLAVYYCQQHYSPPYTFGGGTKLEIKR

SEQ ID NO: 43 - DNA, 324 bp, Mus sp.
  CDS (1)..(324)
gacattgtgatgacccagtctcacaaattcttgtccacatcagttggagacagggtcagtatcacctgcaaggccagtcaggatgtggttactgctgttgcctggtttcaacagaaaccaggacaatctccaaaactactgatttattcggcatcccaccggtacactggagtccctgatcgcttcactggcagtggatctgggacagatttcactttcaccatcatcagtgtgcaggctgaagacctggcagtttattactgtcaacaacattatactactcccacgacgttcggtggaggcaccaagctggacttcagacgg

SEQ ID NO: 44 - protein, 108 aa, Mus sp.
DIVMTQSHKFLSTSVGDRVSITCKASQDVVTAVAWFQQKPGQSPKLLIYSASHRYTGVPDRFTGSGSGTDFTFTIISVQAEDLAVYYCQQHYTTPTTFGGGTKLDFRR

SEQ ID NO: 45 - DNA, 324 bp, Mus sp.
  CDS (1)..(324)
gacactgtgatgacccagtctcacaaattcatatccacatcagttggagacagggtcagcatcacctgcaaggccagtcaggttgtgggtagtgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatataacagctatccgtacacgttcggaggggggaccaagctggaaataaaacgg

SEQ ID NO: 46 - protein, 108 aa, Mus sp.
DTVMTQSHKFISTSVGDRVSITCKASQVVGSAVAWYQQKPGQSPKLLIYWASTRHTGVPDRFTGSGSGTDFTLTISNVQSEDLADYFCQQYNSYPYTFGGGTKLEIKR

SEQ ID NO: 47 - DNA, 324 bp, Mus sp.
  CDS (1)..(324)
gacattgtgatgacccagtctcaaaaattcatgtccacatcagtaggagacagggtcagcgtcacctgcaaggccagtcagaatgtgggtactaatgttgcctggtatcaacacaaaccaggacaatcccctaaaataatgatttattcggcgtcctcccggtacagtggagtccctgatcgcttcacaggcagtggatctgggacacttttcactctcaccatcaacaatgtgcagtctgaagacttggcagagtatttctgtcagcaatataacagctatcctctcacgttcggctcggggacaaagttggaaataaaacgg

SEQ ID NO: 48 - protein, 108 aa, Mus sp.
DIVMTQSQKFMSTSVGDRVSVTCKASQNVGTNVAWYQHKPGQSPKIMIYSASSRYSGVPDRFTGSGSGTLFTLTINNVQSEDLAEYFCQQYNSYPLTFGSGTKLEIKR

SEQ ID NO: 49 - DNA, 342 bp, Mus sp.
  CDS (1)..(342)
cagatccagttggtgcagtctggacctgagctgaagaagcctggagagacagtcaagatctcctgcaaggcttctgggtataccctcacaagctacggaatgaactgggtgaagcaggctccaggaaagggtttaaagtggatgggctggataaacacctacactggagaaccaacatatgctgatgactttaagggacgttttgccttctctttggaaacctctgccagcactgcctttttgcagatcaacaacctcaaaaatgaggacacggctacatatttctgtgtaagacgcgggtttgcttactggggccaagggactctggtcactgtctctgca

SEQ ID NO: 50 - protein, 114 aa, Mus sp.
QIQLVQSGPELKKPGETVKISCKASGYTLTSYGMNWVKQAPGKGLKWMGWINTYTGEPTYADDFKGRFAFSLETSASTAFLQINNLKNEDTATYFCVRRGFAYWGQGTLVTVSA

SEQ ID NO: 51 - DNA, 342 bp, Mus sp.
  CDS (1)..(342)
cagatccagttggtgcagtctggacctgagctgaagaagcctggagagacagtcaagatctcctgcaaggcttctgggtataccttcacaaactctggaatgaactgggtgaagcaggctccaggaaagggtttaaagtggatgggctggataaacacctacactggagagccgacatatgctgatgacttcaagggacggtttgccttctctttggaaacctctgccagctctgcctatttgcagatcagtaacctcaaaaatgaggacacggctacatatttctgtgcaagaaggggttttgtttactggggccaagggactctggtaactgtctctgca

SEQ ID NO: 52 - protein, 114 aa, Mus sp.
QIQLVQSGPELKKPGETVKISCKASGYTFTNSGMNWVKQAPGKGLKWMGWINTYTGEPTYADDFKGRFAFSLETSASSAYLQISNLKNEDTATYFCARRGFVYWGQGTLVTVSA

SEQ ID NO: 53 - DNA, 360 bp, Mus sp.
  CDS (1)..(360)
caggttcagctccagcagtctggggctgagctggcaagacctgggacttcagtgaagttgtcctgtaaggcttctggctacacctttactgactactggatgcagtgggtaaaacagaggcctggacagggtctggagtggattgggactatttatcctggagatggtgatactgggtacgctcagaagttcaagggcaaggccacattgactgcggataaatcctccaaaacagtctacatgcacctcagcagtttggcttctgaggactctgcggtctattactgtgcaagaggggattactacggtagtaattctttggactattggggtcaaggaacctcagtcaccgtctcctca

SEQ ID NO: 54 - protein, 120 aa, Mus sp.
QVQLQQSGAELARPGTSVKLSCKASGYTFTDYWMQWVKQRPGQGLEWIGTIYPGDGDTGYAQKFKGKATLTADKSSKTVYMHLSSLASEDSAVYYCARGDYYGSNSLDYWGQGTSVTVSS

SEQ ID NO: 55 - DNA, 357 bp, Mus sp.
  CDS (1)..(357)
caggtccagttacagcaatctggacctgaactggtgaggcctggggcctcagtgaagatttcctgcaaaacttctggctacgcattcagtggctcctggatgaactgggtgaagcagaggcctggacagggtctagagtggattggacggatttatccgggagatggagatatcatttacaatgggaatttcagggacaaggtcacactgtctgcagacaaatcctccaacacagcctacatgcagctcagcagcctgacctctgtggactctgcggtctatttttgttcgagatgggggacatttacgccgagttttgactattggggccaaggcaccactctcacagtctcctca

SEQ ID NO: 56 - protein, 119 aa, Mus sp.
QVQLQQSGPELVRPGASVKISCKTSGYAFSGSWMNWVKQRPGQGLEWIGRIYPGDGDIIYNGNFRDKVTLSADKSSNTAYMQLSSLTSVDSAVYFCSRWGTFTPSFDYWGQGTTLTVSS

SEQ ID NO: 57 - DNA, 351 bp, Mus sp.
  CDS (1)..(351)
gacgtgaagctggtggagtctgggggaggcttagtgaagcctggagggtccctgaaactctcctgtgaagcctctggattcactttcagtagctataccctgtcttgggttcgccagactccggagacgaggctggagtgggtcgcaaccattagtattggtggtcgctacacctattatccagacagtgtggagggccgattcaccatctccagagacaatgccaagaacaccctgtacctgcaaatgaacagtctgaagtctgaggacacagccatgtattactgtacaagagattttaatggttactctgacttctggggccaaggcaccactctcacagtctcctca

SEQ ID NO: 58 - protein, 117 aa, Mus sp.
DVKLVESGGGLVKPGGSLKLSCEASGFTFSSYTLSWVRQTPETRLEWVATISIGGRYTYYPDSVEGRFTISRDNAKNTLYLQMNSLKSEDTAMYYCTRDFNGYSDFWGQGTTLTVSS

SEQ ID NO: 59 - DNA, 360 bp, Mus sp.
  CDS (1)..(360)
aatgtacagctggtagagtctgggggaggcttagtgcagcctggagggtcccggaaactctcctgtgcagcctctggattcactttcagtaactttggaatgcactgggttcgtcaggctccagagaagggtctggagtgggtcgcatacattcgtagtggcagtggtaccatctactattcagacacagtgaagggccgattcaccatctccagagacaatcccaagaacaccctgttcctgcaaatgaccagtctaaggtctgaggacacggccatgtattactgtgcaagatcctactatgatttcggggcctggtttgcttactggggccaagggactctggtcactgtctctgca

SEQ ID NO: 60 - protein, 120 aa, Mus sp.
NVQLVESGGGLVQPGGSRKLSCAASGFTFSNFGMHWVRQAPEKGLEWVAYIRSGSGTIYYSDTVKGRFTISRDNPKNTLFLQMTSLRSEDTAMYYCARSYYDFGAWFAYWGQGTLVTVSA

SEQ ID NO: 61 - DNA, 324 bp, Homo sapiens
  CDS (1)..(324)
gatatcgtaatgacccagtcccacctgagtatgagtacctccctgggagatcctgtgtcaatcacttgcaaggcctcacaggatgtgagcaccgtcgttgcttggtatcagcagaagcccgggcaatcacccagacgtctcatctactcagcatcataccgttacatcggggtgcctgaccgatttactggctctggcgctggcacagatttcacctttacaattagttccgtccaggccgaagacctggccgtgtactactgccagcagcactacagtcccccatacactttcgggggagggactaagctcgaaatcaaacgt

SEQ ID NO: 62 - protein, 108 aa, Homo sapiens
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 63 - DNA, 324 bp, Homo sapiens
  CDS (1)..(324)
gacattgttatggctcaaagccatctgtctatgagcacatctctgggagatcctgtgtccatcacttgcaaagccagtcaagacgtgtctacagttgttgcatggtatcaacagaagccaggccagtcacccagacggctcatttactcagcttcttaccgatacatcggggtccctgacagatttacaggtagtggggccggtactgacttcacttttactatctcatccgtacaagccgaagacctggcagtatattactgccagcaacattattccccaccctacacattcggcgggggtactaagctggaaattaaacgt

SEQ ID NO: 64 - protein, 108 aa, Homo sapiens
DIVMAQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 65 - DNA, 360 bp, Homo sapiens
  CDS (1)..(360)
caggtacagctcgttcagtccggcgccgaggtagctaagcctggtacttccgtaaaattgtcctgtaaggcttccgggtacacatttacagactactggatgcagtgggtaaaacagcggccaggtcagggcctggagtggattggaacaatatatcccggcgacggcgacacaggctatgcccagaagtttcaaggcaaggcaacccttactgctgataaatcttccaagactgtctacatgcatctgtcttccttggcatctgaggatagcgctgtctattactgtgctaggggggactactatgggtcaaattccctggattactggggccagggcaccagtgtcaccgtgagcagc

SEQ ID NO: 66 - protein, 120 aa, Homo sapiens [sequence omitted]

SEQ ID NO: 67 - DNA, 324 bp, Homo sapiens
  CDS (1)..(324)
gacaccgtgatgacccagtccccctccaccatctccacctctgtgggcgaccgggtgtccatcacctgtaaggcctcccaggtggtgggctccgccgtggcctggtatcagcagaagcctggccagtcccctaagctgctgatctactgggcctccacccggcataccggcgtgcctgaccggttcaccggctccggcagcggcaccgacttcaccctgaccatctccaacgtgcagtccgacgacctggccgactacttctgccagcagtacaactcctacccttacacctttggcggcggaacaaagctggagatcaagcgt

SEQ ID NO: 68 - protein, 108 aa, Homo sapiens
DTVMTQSPSTISTSVGDRVSITCKASQVVGSAVAWYQQKPGQSPKLLIYWASTRHTGVPDRFTGSGSGTDFTLTISNVQSDDLADYFCQQYNSYPYTFGGGTKLEIKR

SEQ ID NO: 69 - DNA, 324 bp, Homo sapiens
  CDS (1)..(324)
gacaccgtgatgacccagtccccctcctccatctccacctccatcggcgaccgggtgtccatcacctgtaaggcctcccaggtggtgggctccgccgtggcctggtatcagcagaagcctggccagtcccctaagctgctgatctactgggcctccacccggcataccggcgtgcctgcccggttcaccggctccggcagcggcaccgacttcaccctgaccatctccaacgtgcagtccgaggacctggccgactacttctgccagcagtacaactcctacccttacacctttggcggcggaacaaagctggagatcaagcgt

SEQ ID NO: 70 - protein, 108 aa, Homo sapiens
DTVMTQSPSSISTSIGDRVSITCKASQVVGSAVAWYQQKPGQSPKLLIYWASTRHTGVPARFTGSGSGTDFTLTISNVQSEDLADYFCQQYNSYPYTFGGGTKLEIKR

SEQ ID NO: 71 - DNA, 351 bp, Homo sapiens
  CDS (1)..(351)
gaggtgcagctggtggagtctggcggcggactggtgaagcctggcggctccctgaggctgtcctgtgaggcctccggcttcaccttctcctcctacaccctgtcctgggtgaggcagacccctggcaagggcctggagtgggtggccaccatctccatcggcggcaggtacacctactaccctgactccgtgaagggccggttcaccatctcccgggacaacgccaagaacaccctgtacctgcagatgaactccctgaagtccgaggacaccgccatgtactactgtacccgggacttcaacggctactccgacttctggggccagggcaccacactgaccgtgtcctcc

SEQ ID NO: 72 - protein, 117 aa, Homo sapiens
EVQLVESGGGLVKPGGSLRLSCEASGFTFSSYTLSWVRQTPGKGLEWVATISIGGRYTYYPDSVKGRFTISRDNAKNTLYLQMNSLKSEDTAMYYCTRDFNGYSDFWGQGTTLTVSS

SEQ ID NO: 73 - DNA, 36 bp, Mus sp.
ggaggatccatagacagatgggggtgtcgttttggc

SEQ ID NO: 74 - DNA, 32 bp, Mus sp.
ggaggatcccttgaccaggcatcctagagtca

SEQ ID NO: 75 - DNA, 32 bp, Mus sp.
  misc_feature (1)..(32): mixed bases are defined as follows H=A+T+C, S=G+C, Y=C+T,  K=G+T,  M=A+C,  R=A+G,  W=A+T,  V = A+C+G, N = A+C+G+T
cttccggaattcsargtnmagctgsagsagtc

SEQ ID NO: 76 - DNA, 35 bp, Mus sp.
  misc_feature (1)..(35): mixed bases are defined as follows H=A+T+C, S=G+C, Y=C+T,  K=G+T,  M=A+C,  R=A+G,  W=A+T,  V = A+C+G, N = A+C+G+T
cttccggaattcsargtnmagctgsagsagtcwgg

SEQ ID NO: 77 - DNA, 31 bp, Mus sp.
  misc_feature (1)..(31): mixed bases are defined as follows H=A+T+C, S=G+C, Y=C+T,  K=G+T,  M=A+C,  R=A+G,  W=A+T,  V = A+C+G, N = A+C+G+T
ggagctcgayattgtgmtsacmcarwctmca

SEQ ID NO: 78 - DNA, 46 bp, Mus sp.
tatagagctcaagcttggatggtgggaagatggatacagttggtgc

SEQ ID NO: 79 - DNA, 21 bp, Mus sp.
atggagtcacagattcaggtc

SEQ ID NO: 80 - DNA, 32 bp, Mus sp.
ttttgaattccagtaacttcaggtgtccactc

SEQ ID NO: 81 - protein, 17 aa, Mus sp.
TIYPGDGDTGYAQKFQG